(19) United States
(12) Patent Application Publication
(10) Pub. No.: US 2020/0368429 A1
(43) Pub. Date: Nov. 26, 2020

(54) The Use of Characteristic Electrochemical Signals for Fluid Identification

(71) Applicants: Bradley Ledden, Fayetteville, AR (US); Greg Lamps, Smyrna, GA (US); Forrest Payne, Fayetteville, AR (US); Emma Sun, Fayetteville, AR (US); SFC FLUIDICS, INC., Fayetteville, AR (US)

(72) Inventors: Bradley LEDDEN, Fayetteville, AR (US); Greg LAMPS, Smyrna, GA (US); Forrest PAYNE, Fayetteville, AR (US); Emma SUN, Fayetteville, AR (US)

(73) Assignee: SFC Fluidics, Inc., Fayetteville, AR (US)

(21) Appl. No.: 16/633,685

(22) PCT Filed: Aug. 17, 2017
(86) PCT No.: PCT/US2018/046843

§ 371 Date: Jan. 24, 2020

Related U.S. Application Data
(60) Provisional application No. 62/546,659, filed on Aug. 17, 2017.

(51) Int. Cl. A61M 5/168; A61M 5/142; A61M 5/172; G01N 33/15

(52) U.S. Cl. CPC A61M 5/16827; G01N 33/15; A61M 5/172; A61M 5/14248

(57) ABSTRACT

A method and device employing the method that identifies a solution based on characteristic electrochemical signal. Many solutions are electroactive, thus producing an electric signal following excitation with an applied potential. An electrochemical sensor can detect these electric signals if it is placed in contact with the fluid. Moreover, a fluid of known composition will produce a characteristic electrochemical signal and this characteristic signal can be used to confirm the presence of that fluid. The use of characteristic electrochemical signal to identify a fluid can be conducted at all scales and in many different fields. A few examples include quality control in food production or pharmaceutical manufacturing or for detection of air bubbles in a fluid stream or for identification of medicament prior to administration via a body worn patch pump/pod.

### CROSS-REFERENCE TO RELATED APPLICATIONS

This application claims priority to U.S. Provisional Appl. No. 62/546,659 filed on Aug. 17, 2017 which is hereby incorporated by reference in its entirety.

### STATEMENT REGARDING FEDERALLY SPONSORED RESEARCH OR DEVELOPMENT

The product development research behind this IP was funded in part by the National Institutes of Health under grant number 1R43DK110972-01.

### BACKGROUND OF THE INVENTION

### Field of the Invention

This invention relates to the identification of a solution based on characteristic electrochemical signal, termed herein as a Fluid Identification Sensor (FIS).

### Description of Related Art

References mentioned in this background section are not admitted as prior art with respect to the present invention.

The first example is U.S. Pat. No. 9,606,037. This patent details a method for determining the composition of a fluid in an intravenous (IV) bag. This method collects sample of fluid at the end of a short dead-end tube and the collected fluid is not passed onto the patient. This type of arrangement would be detrimental in a user-worn pod arrangement, since the pod would require more drug and more hardware, which would increase the size, weight and complexity of the drug delivery device. In a patch pump device, small size and low weight are critical factors in usability.

The second example is U.S. Pat. No. 9,814,846. This details a method for determining that the proper vial is inserted into a pen-type drug delivery device by a circuit printed on the vial. This example confirms drug type by looking at the vial. In the present invention the drug will be loaded from the vial into a reservoir. Even if the proper vial of drug is selected, another confirmation that the correct drug has been loaded into the proper reservoir is required.

The next example is U.S. Pat. No. 9,901,672. This patent outlines an optical identification method for drug in a self-administered pod. This invention deals with the proper identification of patient and drug to ensure the patient-drug paring is correct. Again, this patent is concerned with the identification of proper drug vial and does not confirm that the proper drug has been loaded into a reservoir.

The next example is PCT US2016/056530, which is incorporated by reference as if fully set forth herein. This patent application outlines the physical construction of the similar type of electrochemical sensor as used here and how such a sensor can be used for flow rate determination. The patent application does not outline how such a sensor can be used for fluid identification in a drug delivery device as described herein.

The final example is U.S. Pat. No. 8,187,441, which is incorporated by reference as if fully set forth herein. The patent outlines the use of an electrochemiosmotic pump for fluid delivery (both single-sided and dual-sided pumping). This patent does not outline the use of an electrochemical sensor for fluid identification.

### BRIEF SUMMARY OF THE INVENTION

The invention identifies the type of solution passing an electrochemical sensor by comparing the electrochemical signal generated by the solution to known values. It has particular value for determination of medicament type when multiple medicaments may be dosed to a patient, especially using a body worn patch pump. Medication errors are unfortunately common in medical practice. Patient administered medication suffers the same errors, the consequences of which can be life threatening. Insulin self-administration is moving toward the use of higher concentration insulins as well as dispensing multiple drugs to exert tighter control over blood glucose levels. These drugs could be rapid acting insulin, long acting insulin, glucagon, pramlintide, amylin agonists, or others. The need for multiple medicaments coupled with the increasing use of pumps for continuous subcutaneous infusion can make keeping track of medication loading difficult for some patients. The method presented here allows for a pump or other device to identify that the proper medicament is loaded in the proper reservoir before the pump dispenses drug to the patient.

### BRIEF DESCRIPTION OF THE SEVERAL VIEWS OF THE DRAWINGS

FIG. 1 depicts a block diagram of a dual medicament fluid identification system. The system includes two reservoirs, filled with possibly different medicaments, and two fluid identification sensors.

FIG. 2 depicts characteristic electrochemical signals obtained from insulin carrier using a fluid identification sensor at different applied potentials.

FIG. 3 depicts characteristic electrochemical signals obtained from glucagon carrier using a fluid identification sensor at different applied potentials.

FIG. 4 depicts the electrochemical signal obtained from a fluid identification sensor when the applied potential for glucagon carrier is used with insulin carrier.

FIG. 5 depicts a sequence of events to ready a body-worn pod for use.

FIG. 6 depicts a sequence of events to confirm that fluids are properly loaded into the correct reservoirs.

### DETAILED DESCRIPTION OF THE PREFERRED EMBODIMENTS OF THE INVENTION

Before the present invention is described in further detail, the invention is not limited to the particular embodiments and implementations described, and that the terms used in describing the particular embodiments and implementations are for the purpose of describing those particular embodiments and implementations only, and are not intended to be limiting, since the scope of the present invention will be limited only by the claims.

The most general use of the present invention is identification of a solution flowing through an electrochemical sensor. Many solutions are electroactive, producing an electric signal when they are flowing. An electrochemical sensor can detect these electric signals if it is placed in the path of fluid flow. Moreover, a fluid of known composition will produce a characteristic electrochemical signal and this characteristic signal can be used to identify the presence of that fluid. The use of characteristic electrochemical signal to identify a fluid can be conducted at all scales and in many different fields. A few examples include quality control in food production or pharmaceutical manufacturing or for detection of air bubbles in a fluid stream or for calibration of a fluid identification sensor to a particular production lot. The following discussion details one high need application of the present invention.

Errors in drug delivery are a serious problem. An inline Fluid Identification Sensor (FIS) that can confirm correct drug delivery offers a great benefit. In the hospital (or other medical) setting, this technology can determine if an infusion or injection of the incorrect drug or incorrectly constituted medicament is loaded prior to administration. Preventing such errors is beneficial to patient outcomes. In certain embodiments, the invention is directed to an electrochemical sensor used to identify a drug or drugs being dispensed to the patient.

For industrial or general purposes, this technology can be utilized to determine if incorrect solutions are used in various processes or situations in a similar manner to the disclosed medical applications described in detail herein. Such applications include fluid identification for on-site food testing, biomarker testing, or small-scale industrial processes to name a few.

There is no current standard to ensure that drugs are loaded properly into a patch pump for single or multiple hormone dispense for the treatment of diabetes. One embodiment of multiple hormone dispense is insulin and glucagon. Insulin is potent for lowering circulating levels of blood glucose, while glucagon causes the release of glucose into the blood stream. Another example is insulin and amylin, or amylin analogs. Another example is long-acting and rapid-acting insulins. A final example is rapid acting and long acting medicaments for pain management.

The invention is adapted to several commercial uses, a non-limiting set is set forth here. One embodiment includes a sensor to confirm fluid identity given to a patient from a typical IV set. Such a sensor can identify the proper fluid from a list of commonly administered fluids. Another application includes an FIS in line with the cannula of a drug delivery pump carried on, affixed to, or implanted in the patient. Such a sensor can confirm proper drug delivery to the patient either through a port or other means such as subcutaneous infusion, intravenous infusion, intradermal infusion, or other methods of drug administration. Another application is the administration of pain medicaments delivered to the site of pain. In cases of administering drugs of potential abuse such a sensor indicates the drug was dispensed properly and not excised from the device by another means or that the wrong medicament was not accidentally or intentionally loaded into a patch pump reservoir. A further embodiment includes multiple FISs in line with cannulae that dispense multiple drugs to the patient. The sensors can confirm proper loading of the various drugs into proper reservoirs/lines to ensure proper drug delivery. Another application can confirm that drugs are mixed in the correct ratio when dispensed, either drugs mixed at dispense or drugs loaded pre-mixed where mixture ratio needs confirmation. Another application can confirm the integrity of drug or drugs sold preloaded or loaded into in the pump/pod. An FIS that is broad in scope can be used to identify medicaments from a panel of medicaments with characteristic electrochemical signals.

The explanation of the technology in a dual drug application is provided below. Other embodiments are possible after discussing an embodiment where the two drugs could have opposite action on the patient. For example, insulin lowers blood sugar while glucagon raises blood sugar. Dispensing these two medicaments has the potential to more tightly regulate blood glucose levels in diabetic patients than insulin alone. However, because of their opposing effects, dispensing the opposite drug could have devastating health effects. Correct drug loading is especially important in a device where the user loads drugs. The potential for error increases with more users and the more times that they fill a device. For a multi drug delivery device one potential problem is that the drugs can be cross loaded where drug A is loaded in the reservoir for drug B and vice-versa. The FIS allows for an automated check to make sure the drugs are loaded correctly into the device.

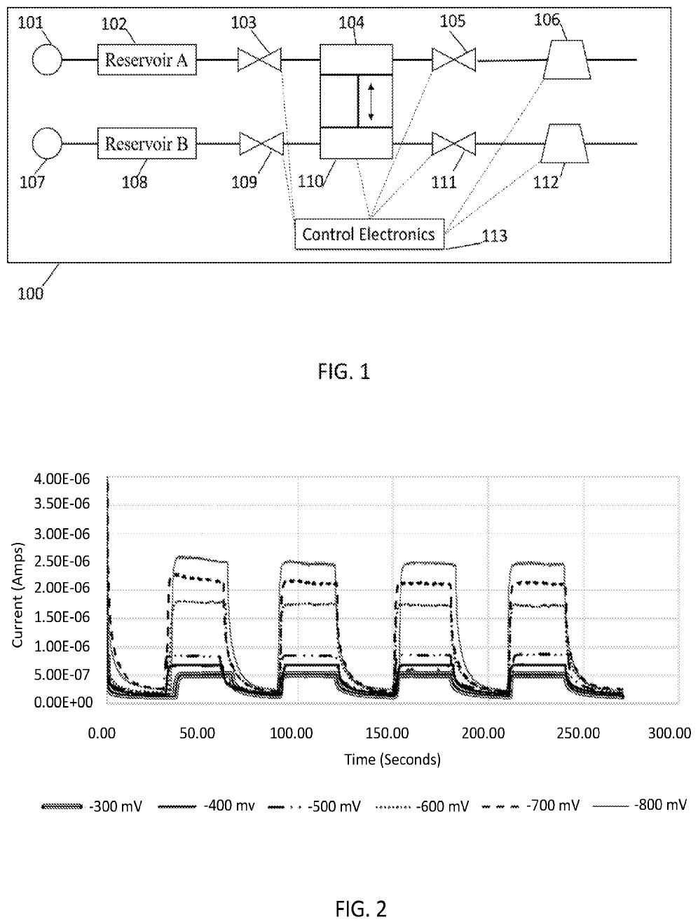

FIG. 1 depicts a block diagram of a dual drug delivery device 100. Drug A is loaded through fill port 101 into reservoir A 102. Drug B is loaded through fill port 107 into reservoir B 108. Each fill port could be clearly labeled to reduce confusion. In addition, the fill ports may be designed so misloading is difficult. However, mistakes could still be possible and the FIS acts as a final check of proper loading. To dispense drug A to the patient, valve 103 is opened and the dual-sided pump draws fluid from reservoir A 102 into chamber 104. Valve 103 is then closed, valve 105 is opened, and the pump dispenses drug A through the fluid identification sensor (FIS) 106 and out through the cannula. This FIS consists of three ring electrodes. A characteristic voltage is applied to one of the electrodes using a potentiostat and a resultant characteristic electrochemical current is obtained. The characteristic current obtained from the solution is compared to stored values in the control electronics 113 to confirm that drug A has been properly loaded into reservoir A 102. If drug B had been inadvertently loaded into reservoir A 102, then upon passage through the FIS 106 a current characteristic of drug A would not be obtained and the control electronics 113 would then sound either an audible alarm or issue some other type of warning message and prevent the pump from dispensing an incorrect drug. Alternatively, if the correct characteristic current for drug A is obtained, the pump then is permitted to dispense the correct drug.

Likewise, drug B is loaded through a fill port 107 to reservoir B 108. To dispense drug B to the patient, the inlet valve 109 opens and the dual-sided pump withdraws drug B from reservoir B 108 into chamber 110. Then the inlet valve 109 closes, outlet valve 111 opens and the pump pushes fluid through FIS 112 and out through the cannula. If the control electronics 113 sense an incorrect characteristic current from the fluid identification sensor 112 then an alarm is issued, preventing the pump from dispensing an incorrect drug (or allowing dispense if the correct drug is identified). Ideally, initial dispenses will be carried out as part of a priming function of the delivery device. Checks for delivery of the correct solution can then occur before either drug is dispensed to the patient. After confirmation of drug identity, the delivery device could then be connected to a port, catheter, or other method for delivery to the patient. One alternative to the above would be the use of two single-sided pumps in place of one dual-sided pump.

FIG. 2 shows typical characteristic currents obtained from the fluid identification sensor (FIS) at several voltages. For this data, insulin carrier solution was passed through the FIS. This solution was made in-house based on a solution formulation on file at the United States Food and Drug Administration for U100 insulin. The data shows the increased current measured when four pulses of flow at 20 μL/min were passed for approximately 30 seconds separated by 30 seconds without flow. The characteristic current during flow is lowest with an applied potential of −300 mV. The next lowest current is with −400 mV applied potential, with −500 mV resulting in a current slightly higher than −400 mV. When the applied potential is changed to −600 mV however, the characteristic current increases significantly. Changing the applied potential to −700 mV and −800 mV results in further but less significant increases in the current measured. A potential of −700 mV is near the middle of the potential window that results in high current as well as stable signal and is a good choice for an operating voltage with insulin carrier.

One possible method to determine if the correct solution is flowing through the FIS 106 or 112 would be to apply a range of voltages and compare the currents obtained to those expected for that solution. In this example, U100 insulin carrier would be expected to produce the characteristic currents at the range of applied potentials shown in FIG. 2.

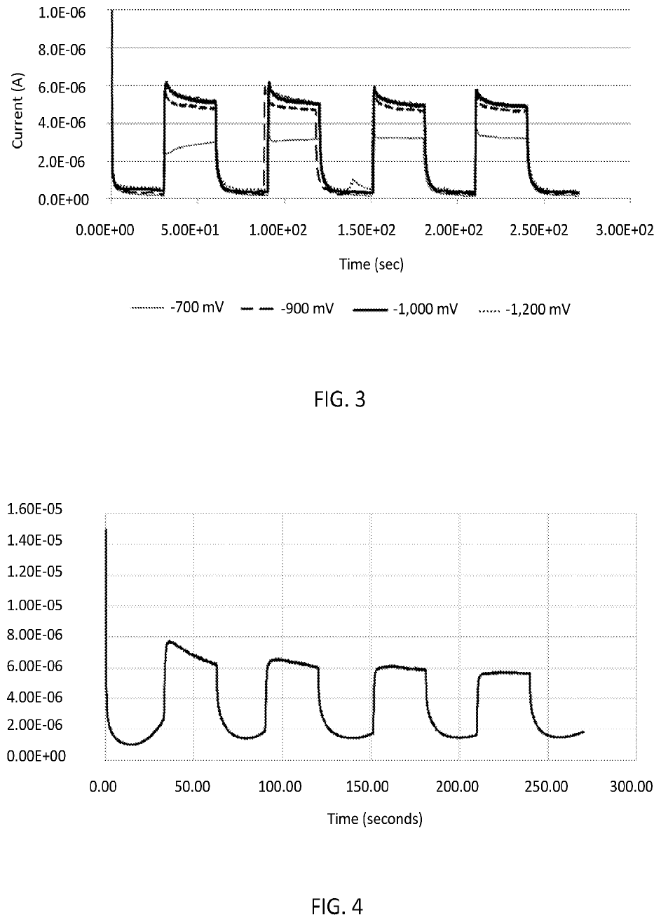

The data in FIG. 3 is of a glucagon carrier solution with the same flow rate, 20 μL/min for 30 seconds with 30 seconds without flow in between the pulses of flow. A glucagon carrier solution uses as a non-aqueous solvent for glucagon. The characteristic current obtained for glucagon carrier is shown for −700 mV applied potential. The signal increases with increase in potential magnitude to −900 mV and then does not rise significantly as potential magnitude is increased further to −1,000 mV and even −1,200 mV. Because the signal does not rise further at a higher potential, a single applied potential of −900 mV is a good choice for this glucagon carrier.

FIG. 4 is an example of incorrect loading of insulin carrier into a fluid identification sensor programmed for glucagon carrier and shows the current obtained with an applied potential of −900 mV. As shown in FIG. 3 at an applied potential of −900 mV, the no flow current should be measured as 0.2 μA and the flow current should be measured as 5 μA for glucagon carrier. However, the measured current in the no-flow condition is approximately 1.8 μA and the flow current is 6 μA. The mismatch between actual and expected currents means that the wrong solution was in the sensor.

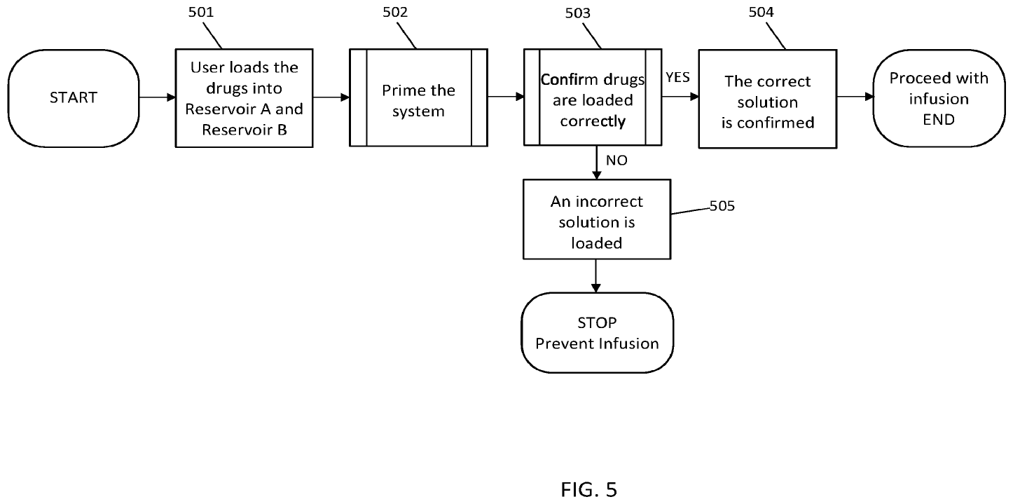

For a multidrug pod there should be a system to confirm functionality of the different components of the pod as well as confirm correct drug loading before the pod is used. A start-up procedure for a two-drug delivery device is outlined briefly in FIG. 5. The user will begin by loading the drugs into Reservoir A 102 and Reservoir B 108 in the first step 501. After loading the drugs, the system will automatically prime itself 502. This step will alternatively cycle the valves 103, 105, 109, and 111 and draw solution from the reservoirs 102, 108 into the pump chambers 104, and 110 depicted in FIG. 1. After priming, the drug delivery pod will confirm the drugs are loaded properly 503. Proper drug loading means the proper drug is loaded into the correct reservoir. If the drugs are loaded into the correct reservoir, the FIS will detect their characteristic currents and the control electronics 113 will permit dispense of the drugs to the patient 504. This will be communicated to the user. If the drugs are loaded into the incorrect reservoirs 505, the FIS will detect that the characteristic currents were not measured and the device will stop, preventing drug infusion. The user will be notified that the drugs were mis-loaded, and the user can dispose of the pod.

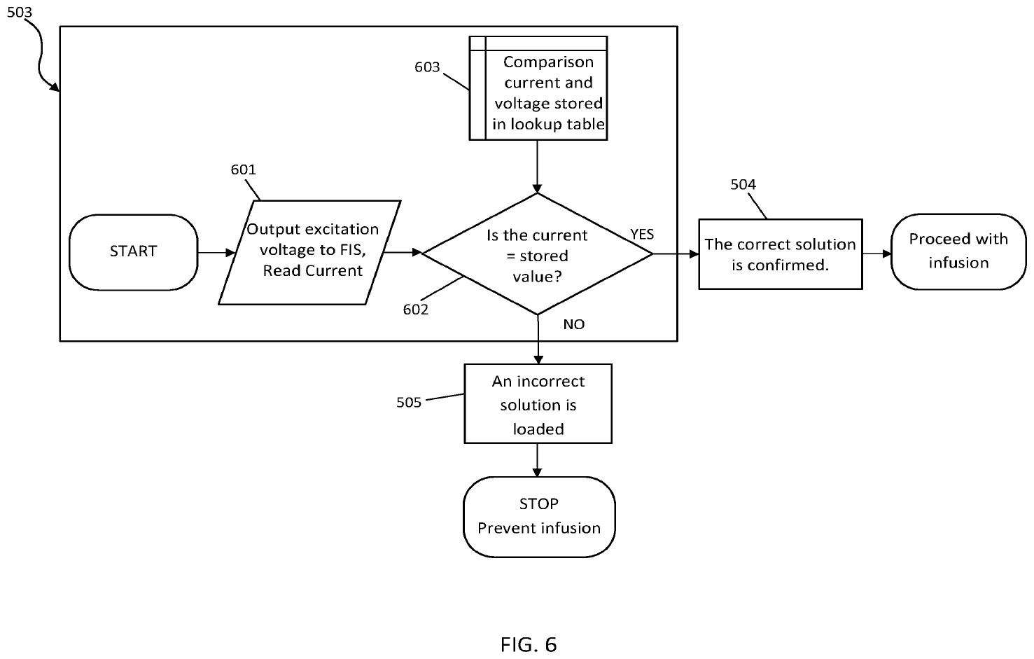

A simple algorithm to determine if the fluids are loaded correctly is shown in FIG. 6. After priming of the fluid into the sensors a potential can be applied and the characteristic current measured 601. The control electronics 113 compare 602 the measured characteristic current with values stored in memory 603. The pod will expect drug A to be loaded into reservoir A and if the measured currents match expected values 504 the pod can continue and proceed with infusion. If the currents do not equal stored values the control electronics 113 will recognize and incorrect solution has been loaded 505 and issue an alarm. Infusion will be prevented in this case.

As a specific, but not restrictive, illustrative example the background signal from insulin carrier is much higher than for glucagon carrier (1.8 μA vs. 0.2 μA) with an excitation voltage of −900 mV. Applying a voltage of −900 mV in step 601 will result in one of two characteristic currents. If the pod is designed to have glucagon in reservoir B 108, then the characteristic current from the dispense confirmation sensor 112 would have an expected value of 0.2 μA stored in memory 603. If the current value obtained during step 601 is different (for instance 1.8 μA which is indicative of insulin carrier) comparison 602 to stored values 603 would indicate the incorrect solution is loaded 505 and infusion would be prevented.

Other mechanisms to determine correct drug loading could be used. One method is to apply a predetermined excitation voltage and record characteristic currents during pumping and during rest. The currents are compared to stored values like those plotted in FIG. 2 and FIG. 3. This method allows confirmation at characteristic voltages for each solution. This technique may require more complex control electronics.

For a drug delivery device designed to dispense insulin and glucagon, each FIS operates best at voltages characteristic of the two solutions. Insulin carrier solution and glucagon carrier solution give optimal signal at two different applied potentials (between reference and working electrode): −700 mV for insulin carrier and −900 mV for glucagon carrier. For such a delivery device, cross loaded drugs could cause significant patient harm since glucagon could be dosed during a state of hyperglycemia further increasing blood glucose levels. Even worse, insulin could be dosed during a state of hypoglycemia. Cross loaded drugs could be the first link in a chain of a potentially fatal sequence of events. After filling with drug, the pod will undergo a start-up sequence which will entail switching the valves and dispensing a few units of each drug to fill the tubing inside the delivery device as well as tubing to the patient. During this start-up procedure drug will also flow through the FIS. One method to determine each drug is passing through the proper fluid path is to compare the current from the FIS to typical currents stored in memory. Another method is to charge each FIS to the same voltage and determine which solution supports the highest current. As an example of this method, glucagon carrier (Drug B) should produce a higher current at −700 mV applied potential than insulin carrier (Drug A) and the control electronics 113 could confirm that the current from FIS for drug B 112 is higher than from the FIS for Drug A 106. Another method would apply a series of voltages to the FIS during pumping and compare the change in signal for insulin carrier from −300 mV up to −900 mV to a look up table. A similar procedure is used for glucagon carrier to confirm its presence. Insulin carrier does not provide a stable signal at the high potentials used for glucagon carrier. Any of these methods allow the pod to confirm the proper drug is loaded in the proper reservoir. In a similar manner changing the voltage and comparing the currents to a look up table determines if the incorrect drug is loaded in a single drug pump.

A similar procedure could be used for other drugs in either a single or multi-drug configuration. Likewise, the sensor could confirm drug presence for applications outside of a pod dispensed medicament.

What is claimed is:

1. A method for identifying a medicament, the method comprising the steps of:
a. passing a solution through at least one sensor comprising at least two electrodes;
b. applying a characteristic voltage to at least one electrode and recording a characteristic current where the current is indicative of the medicament contacting the electrode arrangement; and
c. at a control electronics module, comparing the recorded characteristic current against known characteristic currents and identifying the medicament based on the medicament's known characteristic current.

2. The method of claim 1, wherein the at least one sensor comprises at least one annular ring electrode and the solution is passed through the center of the annular ring electrode.

3. The method of claim 2, wherein the at least one sensor comprises a plurality of annular ring electrodes arranged axially and the solution is passed through the center of each of the plurality of annular ring electrodes.

4. The method of claim 1 wherein identification of the medicament permits further dispense of the medicament.

5. The method of claim 1 wherein lack of identification of the medicament disallows further dispense of the medicament.

6. The method of claim 1 wherein the identification of a panel of medicaments allows for control of the dispense of multiple medicaments.

7. The method of claim 1 wherein the characteristic current is indicative of a solvent in which the medicament is dissolved.

8. The method of claim 1 wherein a characteristic voltage is in the range of +/−1.5 Volts.

9. The method of claim 1 wherein the medicament is selected from the group consisting of the following: insulin, glucagon, pramlintide, amylin agonist, opioid, and opioid analog.

10. The method of claim 1 wherein fluid identification is used to confirm accidental or intentional misloading of the medicament into a reservoir.

11. The method of claim 1 wherein fluid identification is used to confirm the integrity of the medicament loaded into a reservoir.

12. The method of claim 1, wherein the sensor and control electronics module are housed in a body-worn medicament delivery device configured to dispense at least one medicament.

13. The method of claim 1, wherein the medicament comprises a gas.

14. The method of claim 1, wherein the medicament is comprised of segments of fluid classes, segments of liquid separated by segments of gas, or segments of liquid separated by segments of disparate liquids.

15. A method for identifying a fluid, the method comprising the steps of:
a. passing a solution through a sensor comprising at least two electrodes;
b. applying a characteristic voltage to at least one electrode and recording a characteristic current where the current is indicative of the fluid contacting the electrode; and
c. comparing the recorded current against known characteristic currents for multiple fluid types and identifying the fluid type based on the characteristic current.

16. The method of claim 15, wherein the sensor is an annular ring electrode.

17. The method of claim 16, wherein the sensor comprises a plurality of concentric conductive rings separated by a non-conducting material, wherein the fluid flows through a center of the concentric conductive rings.

18. The method of claim 15, wherein the fluid comprises a liquid.

19. The method of claim 15, wherein the fluid comprises a gas.

20. The method of claim 15, wherein the fluid is comprised of segments of fluid classes, segments of liquid separated by segments of gas, or segments of liquid separated by segments of disparate liquids.

21. A fluid identification apparatus for medicament delivery, comprising:
a. a reservoir to receive a medicament;
b. an inlet valve fluidically connected to the reservoir;
c. a dual-sided pump fluidically connected to the inlet valve and comprising a plurality of chambers, wherein the dual-sided pump is configured to draw the medicament from the reservoir through the inlet valve and into one of the plurality of chambers of the dual-sided pump;
d. an outlet valve fluidically connected to one of the plurality of chambers of the dual-sided pump; and
e. a fluid identification sensor fluidically connected to the first outlet valve,
wherein closing of the inlet valve and opening of the outlet valve allows the medicament to flow from one of the plurality of chambers of the dual-sided pump through the outlet valve to the identification sensor.

22. The fluid identification apparatus of claim 21, wherein the identification sensor comprises an annular ring electrode.

23. The fluid identification apparatus of claim 22, wherein the annular ring electrode comprises a plurality of concentric conductive rings separated by a non-conducting material, wherein the medicament passes through a center of the concentric conductive rings.

24. The fluid identification apparatus of claim 21, further comprising:
a. a second reservoir to receive a second medicament;
b. a second inlet valve fluidically connected to the second reservoir, wherein the dual-sided pump is further configured to draw the second medicament from the second reservoir through the second inlet valve and into one of the plurality of chambers of the dual-sided pump;
c. a second outlet valve fluidically connected to one of the plurality of chambers of the dual-sided pump; and
d. a second fluid identification sensor fluidically connected to the second outlet valve,
wherein closing of the second inlet valve and opening of the second outlet valve allows the second medicament to flow from one of the plurality of chambers of the dual-sided pump through the second outlet valve to the second identification sensor.

25. The fluid identification apparatus of claim 24, wherein the second identification sensor comprises an annular ring electrode.

26. The fluid identification apparatus of claim 25, wherein the annular ring electrode comprises a plurality of concentric conductive rings separated by a non-conducting material, wherein the second medicament passes through a center of the concentric conductive rings.

27. A fluid identification apparatus, comprising:
a. a reservoir to receive a fluid;
b. a pump fluidically connected to the reservoir and configured to draw a fluid from the reservoir into the pump; and
c. a fluid identification sensor fluidically connected to the pump,
wherein activation of the pump allows the fluid to flow from the pump to the identification sensor.

28. The fluid identification apparatus of claim 27, wherein the identification sensor comprises an annular ring electrode.

29. The fluid identification apparatus of claim 28, wherein the annular ring electrode comprises a plurality of concentric conductive rings separated by a non-conducting material, wherein activation of the pump causes the drug to pass through a center of the concentric conductive rings.
